(19) United States
(12) Patent Application Publication
(10) Pub. No.: US 2023/0212134 A1
(43) Pub. Date: Jul. 6, 2023

(54) METHOD FOR STARTING UP A REACTOR FOR PREPARING PHTHALIC ANHYDRIDE

(71) Applicant: Clariant International Ltd, Muttenz (CH)

(72) Inventors: Oliver Richter, Germering (DE); Gerhard Mestl, Muenchen (DE); Martin Schoenfelder, Hohenthann (DE); Werner Pitschi, Bruckmuehl (DE)

(21) Appl. No.: 18/009,005

(22) PCT Filed: Jul. 8, 2021
(86) PCT No.: PCT/EP2021/068941

§ 371 Date: Dec. 8, 2022

(30) Foreign Application Priority Data
Jul. 14, 2020 (DE) 10 2020 118 584.7

(51) Int. Cl. C07D 307/89; B01J 8/06; B01J 6/00; B01J 23/22; B01J 21/06

(52) U.S. Cl. CPC C07D 307/89; B01J 6/001; B01J 8/067; B01J 21/063; B01J 23/22; B01J 2208/065; B01J 2208/00194; B01J 2208/00539

(57) ABSTRACT

The present invention relates to a process for starting up a reactor for preparation of phthalic anhydride by the catalytic oxidation of ortho-xylene and/or naphthalene, containing a bed of shaped catalyst bodies and within a temperature-controlled salt bath. The industrial production of phthalic anhydride from ortho-xylene and/or naphthalene is affected by selective gas phase oxidation in a shell and tube reactor cooled with a salt bath, which may contain several thousand reactor tubes. There are 4 to 5 different catalyst layers in each reactor, which are introduced into each reactor successively in axial direction.

The invention relates to a process for starting up a reactor for preparation of phthalic anhydride by the catalytic oxidation of ortho-xylene and/or naphthalene, containing a bed of shaped catalyst bodies and within a temperature-controlled salt bath, comprising the steps of: a) calcining the shaped catalyst bodies, in the presence of air and/or O₂, at a salt bath temperature above 390° C., b) adjusting the temperature of the salt bath to a temperature between 370° C. and 400° C., c) forming a hotspot in the front third of the bed of shaped catalyst bodies in flow direction, by supply of ortho-xylene and/or naphthalene, d) cooling the salt bath to a temperature below 360° C. at a rate of greater than 0.5° C./h and increasing the ortho-xylene and/or naphthalene supply to a loading above 70 g/m³ (STP), at an air flow rate of 2 to 5 m³ (STP)/h, characterized in that, during the feeding with ortho-xylene and/or naphthalene, the absolute reactor inlet pressure does not go below 1435 mbar.

The industrial production of phthalic anhydride (PA) from ortho-xylene (oX) and/or naphthalene (NA) is affected by selective gas phase oxidation in a shell and tube reactor cooled with a salt bath, which may contain several thousand reactor tubes (reactors). There are 4 to 5 different catalyst layers in each reactor, which are introduced into each reactor successively in axial direction. Each catalyst layer constitutes a bed of shaped catalyst bodies; these typically each consist of an inert support ring coated with a catalytically active composition. The active composition in turn typically consists of a mixture of V₂O₅, Sb₂O₃, TiO₂ in the anatase modification, and further promoters. Only after the catalyst has been calcined or preformed in the reactor can the catalyst be started up with ortho-xylene and/or naphthalene. Typically, the initial coolant temperature of the reactor is lowered gradually here from about 390° C., and the carbon loading or feed is increased correspondingly slowly at an air flow rate of 2 to 5 m³ (STP)/h/tube, in order not to exceed a maximum catalyst temperature of about 455° C. and hence to avoid irreversible catalyst deactivation. Equally, the initial salt bath temperature for acceleration of the running-in phase cannot be chosen at an arbitrarily low level, since unconverted underoxidation products (e.g. phthalide, naphthoquinone) otherwise occur to an enhanced degree and adversely affect product quality. Thus, the running-in phase, which generally comprises the period of time from the first feeding of reactant until attainment of the maximum target loading, typically lasts for up to eight weeks or longer. It is particularly disadvantageous in this context that only reduced phthalic anhydride productivity is available during this long running-in phase, since the reactor can at first be operated only with a small amount of hydrocarbon.

The literature discloses various processes that describe how the process parameters of salt bath temperature, hydrocarbon loading and/or air flow rate can be varied during the startup of the catalyst. A summary thereof is given in WO 2014207604 A2. WO 2014207604 A2 likewise discloses a process for preparing carboxylic acids and/or carboxylic anhydrides by gas phase oxidation of aromatic hydrocarbons, in which a gas stream comprising at least one aromatic hydrocarbon and molecular oxygen is passed continuously over a catalyst thermostatted by a heat carrier medium, which comprises lowering the temperature of the heat carrier medium during the startup of the reactor and keeping it constant for at least 24 hours, during which neither the loading of the gas stream with hydrocarbons nor the gas stream volume is increased by more than 3%.

WO 2014207603 A2 discloses a process for preparing phthalic anhydride by gas phase oxidation of aromatic hydrocarbons, in which a gas stream comprising at least one aromatic hydrocarbon and molecular oxygen is passed continuously over a thermostatted catalyst and, after the catalyst has been started up, the feeding of the at least one aromatic hydrocarbon to the catalyst is interrupted temporarily.

WO 2009124946 A1 describes a process for starting up a gas phase oxidation reactor for oxidation of ortho-xylene to phthalic anhydride, said reactor comprising at least one catalyst layer and being temperature-controllable by means of a heat carrier medium, wherein a) the catalyst layer is interrupted by a moderator layer which is less catalytically active than the catalyst layer or is catalytically inactive, b) a gas stream is passed through the reactor with an initial loading of o-xylene and at an initial temperature of the heat transfer medium, c) the loading of the gas stream is increased to a target loading and, in parallel, the temperature of the heat transfer medium is lowered to an operating temperature. The introduction of the moderator layer allows the loading to be increased rapidly and the startup time to be shortened.

None of the existing startup processes enables distinct acceleration of the running-in phase to shorter than two weeks.

It was thus an object of the invention to provide a process for startup with which the running-in phase is distinctly accelerated, without simultaneously adversely affecting the catalyst performance of the catalyst that has undergone full startup (product yield, by-product formation). The accelerated running-in phase distinctly increases phthalic anhydride production capacity compared to a catalyst started up by the customary methods in the first weeks and months.

The object of the invention was achieved by the provision of a process for starting up a reactor for preparation of phthalic anhydride by the catalytic oxidation of ortho-xylene and/or naphthalene, containing a bed of shaped catalyst bodies and within a temperature-controlled salt bath, comprising the steps of:
a) calcining the shaped catalyst bodies, in the presence of air and/or O₂, at a salt bath temperature exceeding 390° C.,
b) adjusting the temperature of the salt bath to a temperature between 370° C. and 400° C.,
c) forming a hotspot in the front third of the catalyst bed in flow direction, by feeding in ortho-xylene and/or naphthalene,
d) cooling the salt bath temperature to a salt bath temperature below 360° C. at a rate of greater than 0.5° C./h and increasing the feed of ortho-xylene and/or naphthalene to a loading exceeding 70 g/m³ (STP), at an air flow rate of 2 m³ (STP)/h to 5 m³ (STP)/h,
characterized in that, during the feeding with ortho-xylene and/or naphthalene, the absolute reactor inlet pressure does not go below 1435 mbar.

The unit [m³ (STP)] relates to one standard cubic meter under standard conditions, i.e. a standard pressure of 1013.25 mbar and the standard temperature of 273.15 K to DIN 1343.

The process of the invention is employed for startup of any salt bath reactor for preparation of phthalic anhydride by oxidation of ortho-xylene and/or naphthalene. It is preferable that the process of the invention is used for startup of a typical shell and tube reactor for preparation of phthalic anhydride. In such a typical commercial shell and tube reactor, the reactor tubes are present individually in a vessel cooled with a salt bath. In the context of this invention, the reactor means the individual reactor tubes in the salt bath, meaning that all details of the flow of gases relate to a single reactor tube, i.e. a single reactor. The two openings of the respective reactor form a reactor inlet for the reactant gas and a reactor outlet for the product gas, such that there are a gas inlet side, a gas outlet side and a gas flow direction. The tubular reactor in each case, for example, has an internal diameter in the range from 10 to 50 mm, preferably between 20 and 40 mm. The tubular reactors have a tube length in the customary ranges, for example 2 to 5 m. The tube length corresponds here to the length of the reactor tube filled with the shaped catalyst bodies.

The reactor is within a temperature-controllable salt bath which generally uses, as heat carrier medium, a salt melt, for example a eutectic mixture of NaNO₂ and KNO₃. The salt bath or salt melt can be heated, i.e. heated in a controlled manner to temperatures of up to 460° C., before beginning to break down and releasing toxic nitrogen oxides. The exothermic reaction and any different catalyst layers present result in formation of a temperature profile in axial direction in the reactor. The temperature figures for performance of the process of the invention for startup therefore relate to the salt bath temperature, which is typically measured, for the purpose of regulation and control, downstream of the circulation pump before reentry into the salt bath.

The shaped catalyst bodies consist of an inert support body and an active composition applied thereto. The shaped catalyst bodies of the catalyst layers are typically produced by applying a thin layer of the active composition to the inert support body. The production of typical shaped catalyst bodies for preparation of phthalic anhydride by oxidation of ortho-xylene and/or naphthalene is detailed, for example, in EP 3134394 A1 and EP 3008050 A1.

The active composition may, as well as vanadium, have numerous promoters, for example alkali metals and/or alkaline earth metals, antimony, phosphorus, iron, niobium, cobalt, molybdenum, silver, tungsten, tin, lead, zirconium, copper, gold and/or bismuth, and mixtures of two or more of the above components. In one embodiment, the shaped catalyst bodies in the individual catalyst layers differ in their active composition. Typical active compositions are detailed, for example, in EP 3134394 A1 and EP 3008050 A1.

Startup of the reactor is understood here to mean the phase after the introduction of the shaped catalyst bodies into the reactor until normal production operation at target loading. The startup thus includes the running-in phase, which is typically understood to mean the period of time from the first feeding of reactants until normal production operation. During the process of the invention for startup, process steps a) to d) of the invention are performed successively; preferably, the process of the invention consists of these process steps.

The shaped catalyst bodies are first calcined in a calcining step a), in order to burn off the binder present in the active composition and to (pre)form the active composition. The calcination is effected in the presence of air and/or oxygen, while increasing the salt bath temperature from, for example, room temperature to above 390° C., preferably above 400° C. most preferably 430° C.; the calcination temperature is preferably in the range between 390° C. and 460° C., more preferably in the range between 400° C. and 440° C. During the calcining step a), a stream of air or oxygen is passed through the reactor; the gas flow rate or air flow rate is of no great relevance here, but must be sufficiently high to assure complete burnoff of the binder and sufficient preforming of the catalyst. The gas flow rate or air flow rate may, for example, be between 0.02 and 4.5 m³ (STP)/h, preferably 0.05 to 1.5 m³ (STP)/h. The calcining step a) should be conducted for more than 6 h, preferably for at least 24 h; the calcining step a) may also be performed for longer, for example for more than 48 h or more than 72 h.

In a step b) that follows the calcining step a), the salt bath temperature is heated from the calcining temperature employed to a salt bath temperature between 370° C. and 400° C., i.e. the salt bath temperature is kept within this range. In general, the calcining temperature is higher than the temperature in step b); therefore, the adjustment of temperature is generally associated with cooling. This adjustment of temperature or cooling is necessary before commencing with the feeding of ortho-xylene and/or naphthalene, in order that the shaped catalyst bodies in the reactor are within the correct temperature range and in particular are not too hot when the oxidation reaction sets in. In this context, however, the rate at which the temperature is adjusted is of no relevance, and so, by way of example, lowering can be conducted after the calcination in step a) within a few minutes from within up to 48 h. It is likewise possible by way of example to effect cooling within 6 h to 48 h while maintaining the air/oxygen flow rate from calcining step a).

In a step c) that follows step b), a temperature maximum (“hotspot”) must be formed in the front third of the catalyst bed. For this purpose, as soon as the temperature has gone below about 400° C., it is possible to commence passage of a stream of ortho-xylene and/or naphthalene through the reactor, i.e. to start up the reactor. The salt bath temperature in step c) is kept within a temperature range between 370° C. and 400° C. for a period of time between 1 h and 220 h, preferably 80 h to 180 h, and more preferably is kept at a virtually constant temperature. It is also preferable that, during step c), the temperature is kept constant for more than 50 h, preferably for more than 100 h, or for between 10 h and 200 h.

The hydrocarbon feed rate during step c) is preferably more than 5 g/m³ (STP), more preferably more than 20 g/m³ (STP) or 25 g/m³ (STP), very especially preferably between 10 g/m³ (STP) and 40 g/m³ (STP), an air flow rate between 2 m³ (STP)/h and 4.5 m³ (STP)/h, preferably between 3 m³ (STP)/h and 4.2 m³ (STP)/h. Typically, under these conditions, the “hotspot” is formed in the front portion of the reactor within 6 h to 72 h.

In order that the reactor can move into normal production operation with sufficiently high production capacity, the salt bath temperature has to be lowered further in a step d) that follows step c), for example below 360° C. In step d), the cooling phase, the salt bath temperature is cooled down continuously or stepwise at a rate of more than 0.5° C./h, until a salt bath temperature below 360° C. is attained. It is preferable that the cooling in step d) is effected at a rate of more than 0.7° C./h, preferably more than 1° C./h. It is also preferable that the cooling in step d) is cooled down at a rate of 0.5° C./h to 10° C./h, preferably of 1° C./h to 10° C./h, most preferably 1° C./h to 3° C./h.

At the same time, in step d), the feed of ortho-xylene/naphthalene is increased to a value corresponding to normal production conditions, i.e. a value greater than 70 g/m³ (STP), at an air flow rate of 2 to 4.5 m³ (STP)/h.

During normal production operation, about 2 to 4.5 m³ (STP)/h of air is passed through the reactor with a loading of 30 to 110 g ortho-xylene/m³ (STP) of air (target loading) at a salt bath temperature below 360° C. It is possible here to retain the elevated reactor inlet pressure of the invention or else to reduce the reactor inlet pressure to customary values in the range between 1300 mbar and 1400 mbar.

Typically, during step d), the feed of ortho-xylene/naphthalene is increased stepwise, but this can also be effected continuously. Preference is given to an increase in the ortho-xylene/naphthalene feed in which the target loading is achieved by increasing the feed at regular intervals such that the target loading is achieved when the cooling in step d) is complete. This can be effected uniformly, but in practice is effected stepwise, preferably with respective increases in the ortho-xylene and/or naphthalene feed in approximately uniformly distributed time intervals during the cooling phase, such that the desired ortho-xylene and/or naphthalene feed in production operation is obtained at the end of the cooling phase. The exact distribution of the time intervals in which the ortho-xylene and/or naphthalene feed is increased is uncritical, but the increase in ortho-xylene and/or naphthalene feed should not exceed 10 g/m³ (STP) per hour, preferably 5 g/m³ (STP) per hour. It is preferable that the level of the feed with ortho-xylene and/or naphthalene is adjusted such that a temperature of the shaped catalyst bodies of 455° C. is not exceeded, since the shaped catalyst bodies would otherwise be irreversibly damaged.

The ortho-xylene and/or naphthalene feed in steps b), c) or d) should each case only be sufficiently high that the temperature of the shaped catalyst bodies does not exceed 455° C., since the shaped catalyst bodies would in that case be damaged. The temperature of the shaped catalyst bodies should be equated with the temperature in the reactor, and is typically ascertained by means of thermocouples introduced. Particularly during the cooling phase in step d), the increase in the ortho-xylene/naphthalene feed from the feed being applied at the end of step c) to the target loading above 70 g/m³ (STP) can suitably be effected in such a way that the temperature of the shaped catalyst bodies does not exceed 455° C. For example, the person skilled in the art, with given air flow rates, can increase the xylene and/or naphthalene feed stepwise only to such an extent that the temperature of the shaped catalyst bodies of 455° C. is not attained.

In industrial processes for preparation of phthalic anhydride by the oxidation of ortho-xylene/naphthalene, an air flow rate per reactor of 2 m³ (STP)/h to not more than 4.5 m³ (STP)/h, usually 3 m³ (STP)/h to 4 m³ (STP)/h, should generally be applied during startup and operation. In all executions of the invention, it is therefore advantageous to pass an air flow rate of 2 m³ (STP)/h to 4.5 m³ (STP)/h or 3 m³ (STP)/h to 4 m³ (STP)/h through the reactor during steps a) to d), but at least during steps b) to d). Depending on the process step, this air stream may then be loaded with the appropriate defined amount of ortho-xylene/naphthalene, i.e. ortho-xylene/naphthalene are in the gaseous state and are passed through the reactor in a mixture with the air.

According to the invention, the absolute reactor inlet pressure during the feeding with ortho-xylene and/or naphthalene in steps a) to d) or at least during steps b) to d) is not less than 1435 mbar, preferably greater than 1450 mbar, more preferably greater than 1470 mbar. The absolute reactor inlet pressure may be within a range between 1435 mbar and 2000 mbar, preferably between 1450 mbar and 1600 mbar. The pressure figure here corresponds in each case to the absolute backpressure in flow direction at the reactor inlet upstream of the shaped catalyst bodies. This elevated reactor inlet pressure is achieved, for example, via the configuration of the catalyst bed, or of the shaped catalyst bodies. A dense packing of the shaped catalyst bodies generally leads to elevated pressure drop, such that the use of small shaped catalyst bodies results in an elevated reactor inlet pressure. It is particularly preferable that the elevated reactor inlet pressure is maintained by the pressure drop properties of the bed of shaped catalyst bodies, the reactor outlet pressure and/or the air flow rate. In addition, the reactor outlet pressure is influenced by plant components that are downstream of the reactor in flow direction, for example valves, baffle plates, pipelines, pipe bends, gas coolers, separation apparatuses for phthalic anhydride.

There are preferably at least two catalyst layer is in the reactor, with one catalyst layer constituting a bed of uniform shaped catalyst bodies in the reactor. If the reactor takes the form of a vertical tube, the length of the respective catalyst layer is the same as the fill height of the respective catalyst layer.

The first catalyst layer faces the gas inlet side of the reactor; it is followed directly in gas flow direction by the at least second catalyst layer, consisting of shaped catalyst bodies that differ from the shaped catalyst bodies in the first catalyst layer. A typical commercial reactor for preparation of phthalic anhydride has 4 to 5 catalyst layers. The individual catalyst layers here may have different voidage.

Voidage LG as a property of a catalyst layer is calculated by equation Eq.1.

$$\text{LG} = 100 * \left({\text{V}}_{\text{KL}} - {\text{V}}_{\text{FK}}\right) / {\text{V}}_{\text{KL}} = 100 * {\text{V}}_{\text{LR}} / {\text{V}}_{\text{KL}}$$

V_{KL} = total volume of the catalyst layer in the reactor, i.e. Π * D²/4 * Lₓ (Lₓ= length of the respective catalyst layer x in gas flow direction, D = internal diameter of reactor)
V_{LR} = volume of the empty space within the filled catalyst layer
V_{FK} = volume of the catalyst bodies within the filled catalyst layer

In order to form the higher voidage in the first catalyst layer, the catalyst bodies of the first and second catalyst layers may differ in one or more geometric dimensions and/or in their geometric shape. Preferred geometric shapes are the cylindrical and annular shapes. The geometric dimensions, corresponding, for example, to the height, length and width of the catalyst body, are preferably chosen so as to result in a volume of the shaped catalyst body in the range from 0.05 to 0.5 cm³.

Preferably, the first catalyst layer is disposed at the gas inlet side and the second catalyst layer downstream of the first catalyst layer in gas flow direction, and the length of the first catalyst layer in gas flow direction is less than the length in gas flow direction of the second catalyst layer, and the first catalyst layer has higher voidage compared to the second catalyst layer.

Preferably, the higher voidage of the first catalyst layer compared to the second catalyst layer arises because the catalyst bodies in the first catalyst layer differ from the catalyst bodies in the second catalyst layer in one or more geometric dimension(s) and/or in their geometric shape.

Preferably, the catalyst bodies of the first catalyst layer and the catalyst bodies of the second catalyst layer are annular, and the catalyst bodies of the second catalyst layer have a lower geometric dimension than those of the first catalyst layer.

Preferably, the voidage of the first catalyst layer is at least 0.6% higher than the voidage of the second catalyst layer.

More preferably, the voidage of the first catalyst layer is at least 1.5% higher than the voidage of the second catalyst layer.

Preferably, the shaped catalyst bodies of the first catalyst layer, based in each case on the mass of the catalyst bodies, have a higher active composition loading than the catalyst bodies of the second catalyst layer.

In particular, it is preferable that the shaped catalyst bodies of the second catalyst layer have lower voidage, as described in EP 3008050 A1; in particular, the second catalyst layer may have a voidage below 65%. Particularly preferred catalyst layer arrangements are detailed in EP 3134394 A1 and EP 3008050 A1; these advantageous executions that are described in general terms therein and in the examples shall form part of the present disclosure by reference.

In a preferred execution of the invention, the salt bath temperature is heated in a step a) to above 390° C. to 460° C. for more than 6 h, and air is passed through the reactor at a throughput of 0.02 m³ (STP)/h to 4.5 m³ (STP)/h. In a step b) that follows step a), the salt bath temperature is adjusted to 370° C. to 400° C. In a step c) that follows step b), at an air flow rate of 2 to 4.5 m³ (STP)/h, an ortho-xylene and/or naphthalene feed of greater than 20 g/m³ (STP) is applied, while simultaneously keeping the temperature virtually constant within the temperature range between 370° C. and 400° C. for 1 h to 220 h. In step d) that follows step c), the salt bath temperature is cooled down at a rate of between 0.5° C./h and 10° C./h until a temperature below 360° C. is attained; simultaneously, during the cooling, the ortho-xylene and/or naphthalene feed is in each case increased stepwise only to such an extent that the maximum temperature of the shaped catalyst bodies in the reactor remains below 455° C.

In a further preferred execution of the invention, the salt bath temperature is heated in a step a) for more than 24 h to above 400° C. to 440° C., and air is passed through the reactor at a flow rate of 0.05 to 1.5 m³ (STP)/h. In a step b) that follows step a), the salt bath temperature is lowered to 370° C. to 400° C. In a step c) that follows step b), at an air flow rate of 3 to 4.2 m³ (STP)/h, an ortho-xylene-/naphthalene feed of 10 to 40 g/m³ (STP) is applied, while simultaneously keeping the temperature approximately constant within the temperature range between 370° C. to 400° C. for 80 h to 180 h. In step d) that follows step c), the salt bath temperature is cooled down at a rate of between 1° C./h and 10° C./h until a temperature below 360° C. is attained; simultaneously, during the cooling, the ortho-xylene and/or naphthalene feed is in each case increased only to such an extent that the maximum temperature of the shaped catalyst bodies in the reactor remains below 455° C.

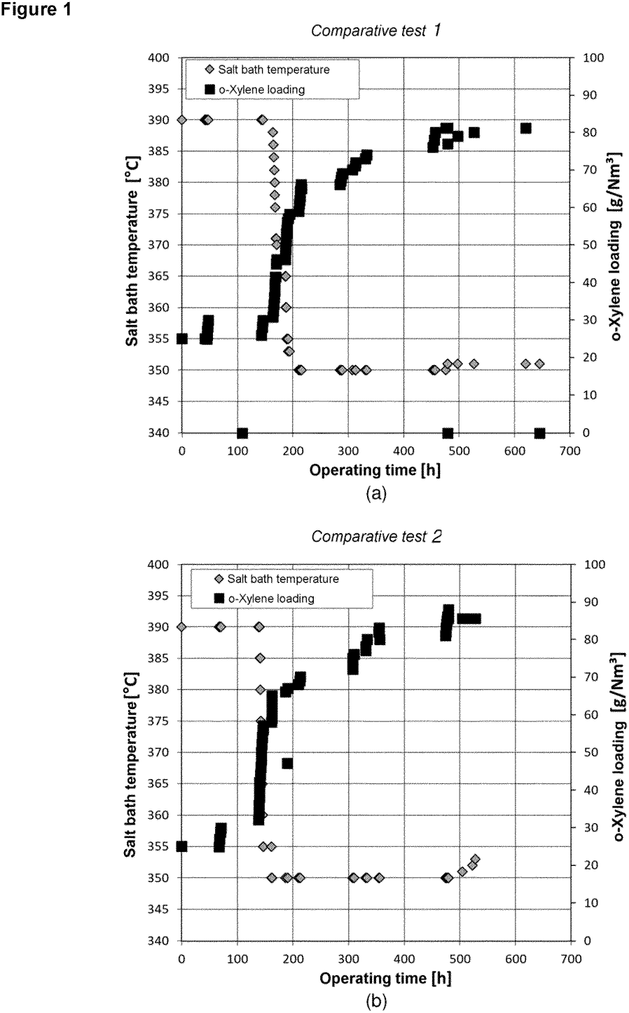

FIG. 1: Evolution with time of salt bath temperature and ortho-xylene feed during process steps b) and c) (absolute reactor inlet pressure in each case < 1435 mbar): (a) comparative test 1, (b) comparative test 2.

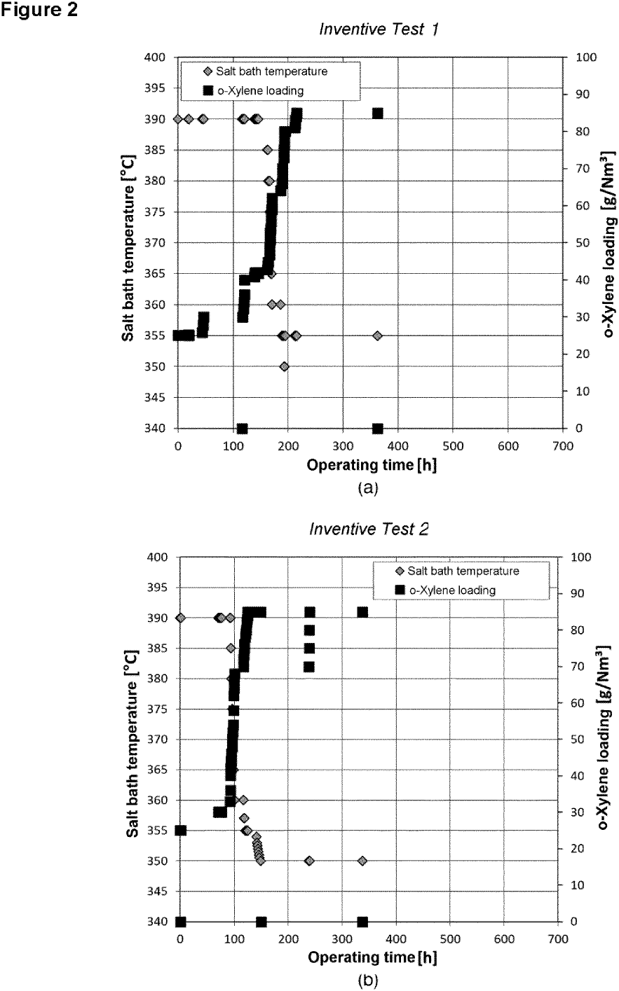

FIG. 2: Evolution with time of salt bath temperature and ortho-xylene feed during process steps b) and c) (absolute reactor inlet pressure in each case > 1435 mbar): (a) inventive test 1, (b) inventive test 2.

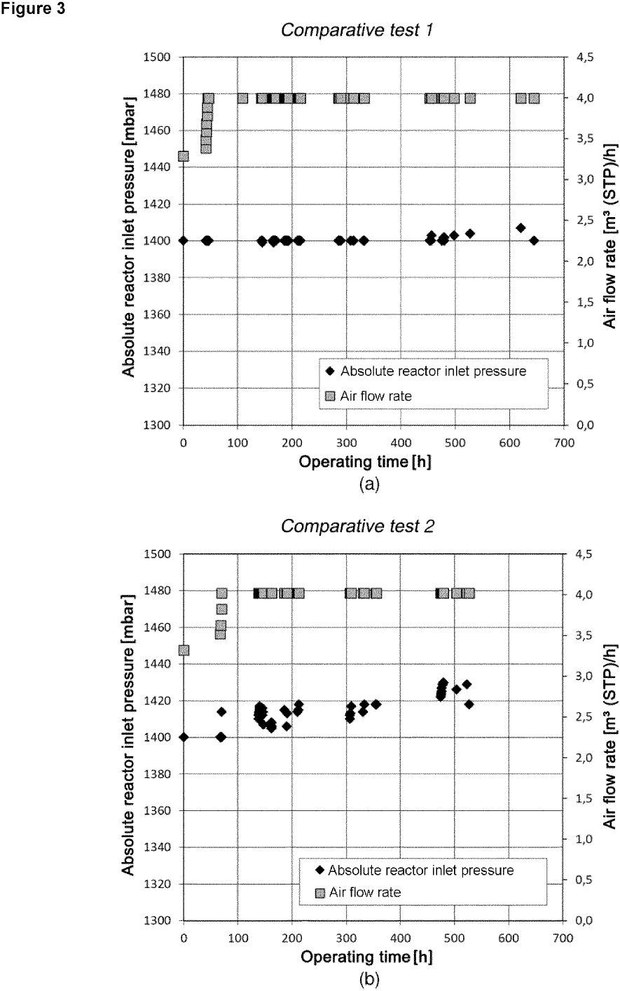

FIG. 3: Evolution with time of air flow rate and absolute reactor inlet pressure during process steps b) and c) (absolute reactor inlet pressure in each case <1435 mbar): (a) comparative test 1, (b) comparative test 2.

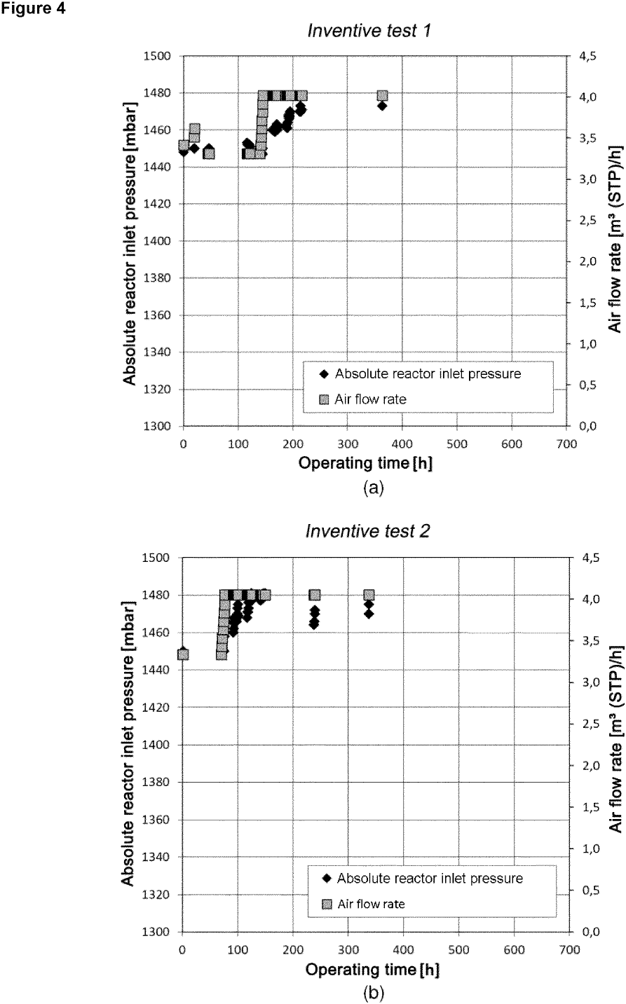

FIG. 4: Evolution with time of air flow rate and absolute reactor inlet pressure during process steps b) and c) (absolute reactor inlet pressure in each case >1435 mbar): (a) inventive test 1, (b) inventive test 2.

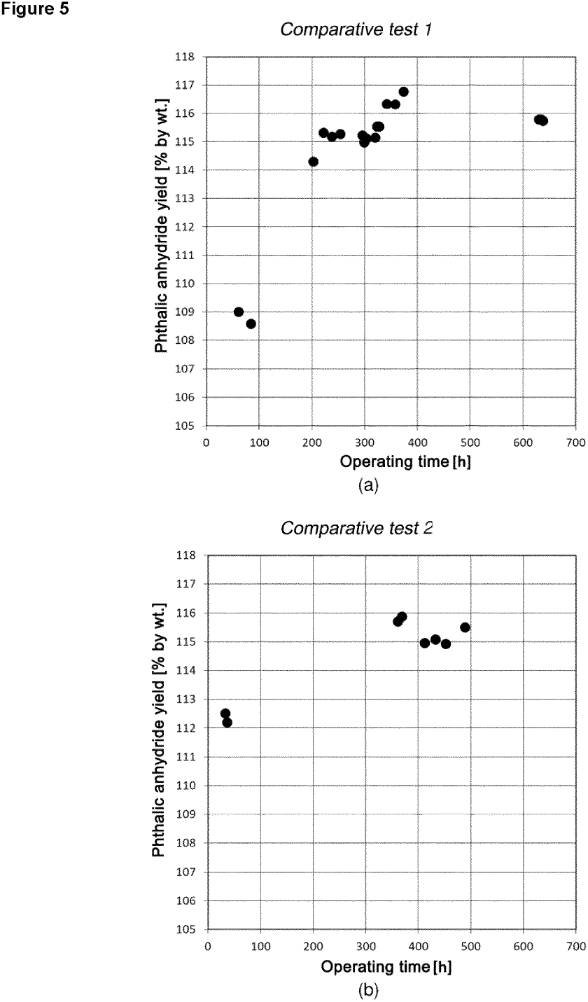

FIG. 5: Evolution with time of phthalic anhydride yield during process steps b) and c) (absolute reactor inlet pressure in each case <1435 mbar): (a) comparative test 1, (b) comparative test 2.

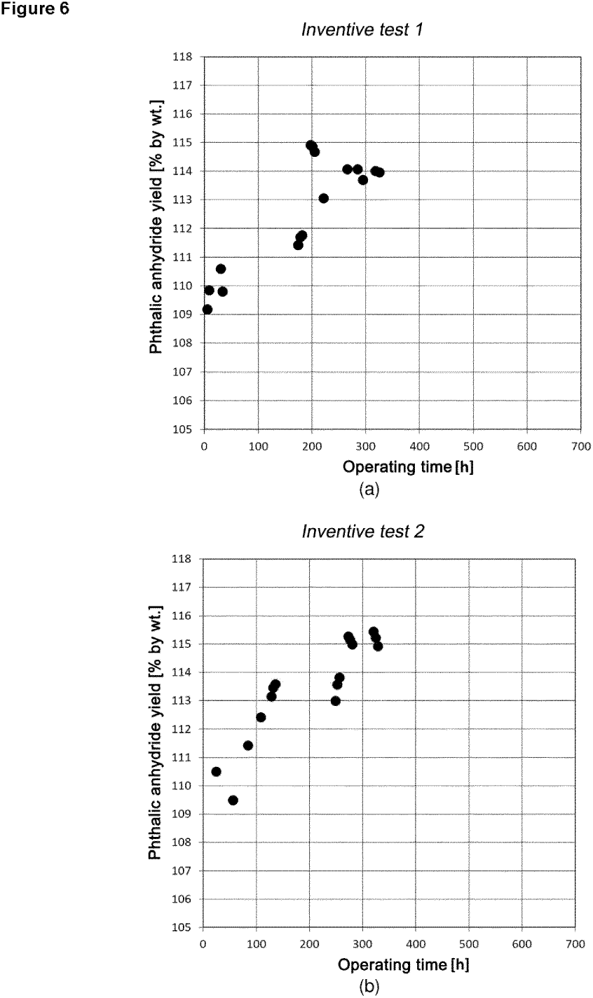

FIG. 6: Evolution with time of phthalic anhydride yield during process steps b) and c) (absolute reactor inlet pressure in each case >1435 mbar): (a) inventive test 1, (b) inventive test 2.

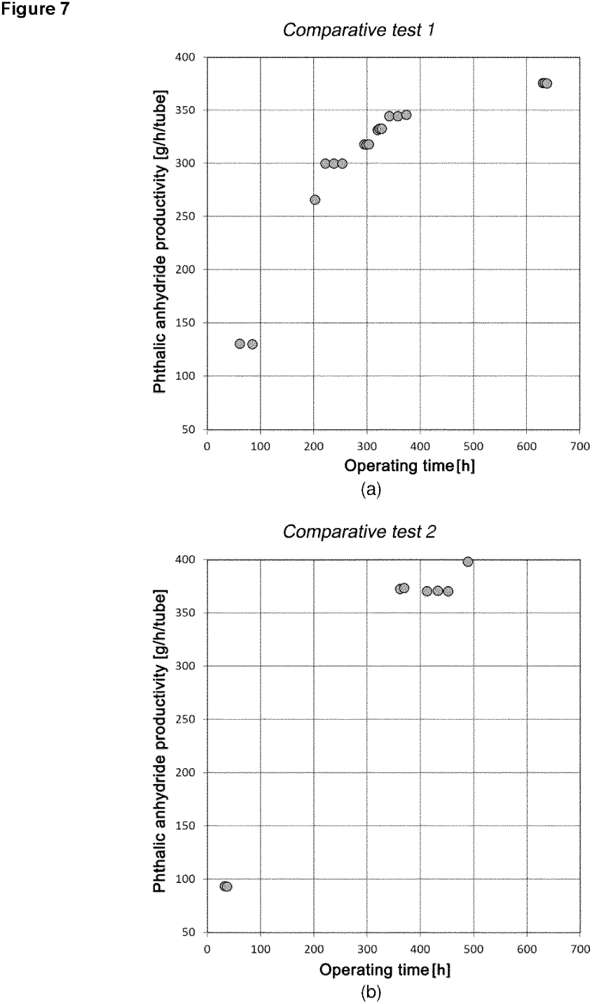

FIG. 7: Evolution with time of phthalic anhydride productivity during process steps b) and c) (absolute reactor inlet pressure in each case <1435 mbar): (a) comparative test 1, (b) comparative test 2.

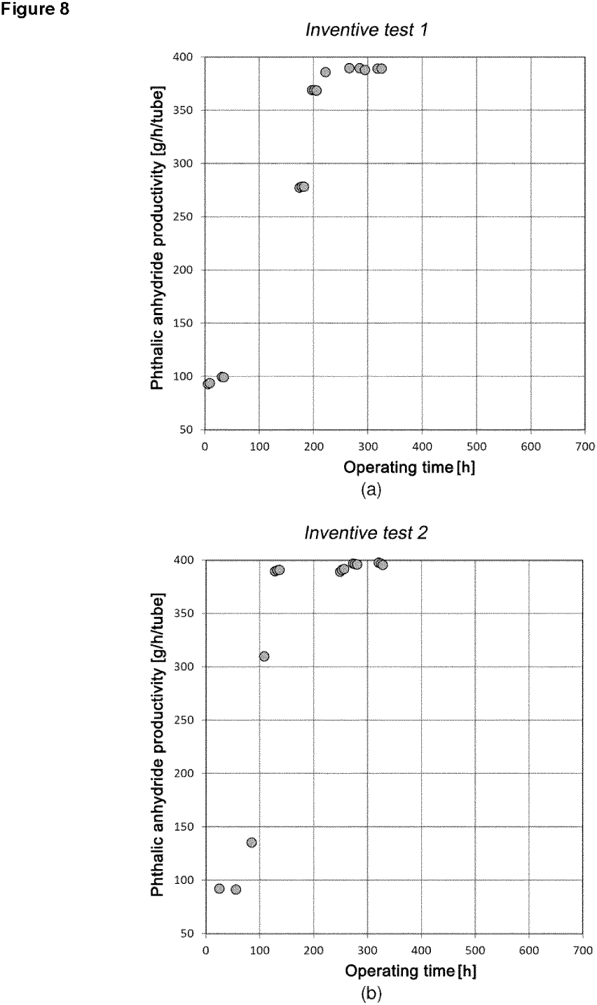

FIG. 8: Evolution with time of phthalic anhydride productivity during process steps b) and c) (absolute reactor inlet pressure in each case >1435 mbar): (a) inventive test 1, (b) inventive test 2.

### EXAMPLES

Catalytic measurements were conducted on 4 identical catalyst layer arrangements of shaped catalyst bodies, with variation of the absolute reactor inlet pressure between < 1435 mbar (noninventive comparative examples) and >1435 mbar (inventive examples).

For synthesis of the shaped catalyst bodies used, two different types of steatite rings were used as shaped bodies, designated 8×6×5 ring and 6×5×4 ring. The nomenclature of the geometric dimensions of the rings corresponds to external diameter (De) [mm] x height (H) [mm] x internal diameter (Di) [mm]. The uncoated shaped bodies were introduced into a coating apparatus and coated homogeneously with the active composition, as described in DE 19709589 A1. During the coating operation, an aqueous suspension of the active components (TiO₂, V₂O₅, promoters) and an organic binder (vinyl acetate/ethylene copolymer) was sprayed onto the fluidized inert support heated to 70° C. via multiple nozzles until the desired active composition layer had formed. Table 1 shows an overview of the shaped catalyst bodies produced and the respective active composition.

For formation of the arrangements of 4 catalyst layers for the respective catalytic measurement, the shaped catalyst bodies were introduced into a salt bath-cooled tube having internal diameter 25 mm and length 4 m. Tables 2 to 5 show an overview of the respective virtually identical fillings as used in the different catalytic tests. For in situ calcination and preforming in process step a), 0.02 to 0.03 m³ (STP)/h of air was passed in each case through the shaped catalyst bodies in the tube at salt bath temperature 410° C. for more than 48 h. In a centered arrangement within the tube was a 3 mm thermowell with an installed tensile element for temperature measurement.

In process step b), before the reactor was started up, the salt bath temperature was cooled from the calcining temperature to 390° C.

At the start of the respective catalytic measurement in step c) (operating time 0 h), at a salt bath temperature of 390° C., air was passed through the tube from the top downward at an air flow rate of 3.3 m³ (STP)/h and a feed rate of 25 g ortho-xylene/m³ (STP) (ortho-xylene purity > 98%). The absolute reactor inlet pressure here was adjusted (in accordance with the invention) by means of a valve downstream of the reactor to a value of < 1435 mbar (noninventive) or > 1435 mbar (inventive). After an operating time of at least 41 h, the air flow rate was in each case increased from 3.3 m³ (STP)/h to 4.0 m³ (STP)/h, at first keeping the salt bath temperature constant at 390° C. After an operating time of 164 h in comparative example 1, after an operating time of 140 h in comparative example 2, after an operating time of 162 h in inventive example 1, and after an operating time of 94 h in inventive example 2, in process step d), the salt bath temperature in each case was lowered stepwise from 390° C. to 350 to 355° C. within 46 h in comparative example 1, within 21 h in comparative example 2, within 30 h in inventive example 1, and wherein 56 h in inventive example 2. At the same time, the feed of ortho-xylene in the air was increased to the maximum possible extent, but such that a temperature of the shaped catalyst bodies of 455° C. was not exceeded.

In the catalytic measurements of the invention (absolute reactor inlet pressure > 1435 mbar), it was possible to achieve a maximum feed rate of 80 to 85 g ortho-xylene/m³ (STP) of air directly with attainment of the minimum salt bath temperature of 350° C. to 355° C. In the noninventive comparative examples (absolute reactor inlet pressure < 1435 mbar), by contrast, the feed rate was limited to 65 g ortho-xylene/m³ (STP) of air with attainment of the minimum salt bath temperature of 350° C. to 355° C.; an increase in the feed rate above that would have increased the temperature of the shaped catalyst bodies to more than 455° C. Only after operation for a further 250 h was it possible to achieve a feed rate of 80 to 85 g ortho-xylene/m³ (STP) of air without further lowering the salt bath temperature.

A detailed overview of the evolution with time of all relevant process parameters in the comparative tests (absolute reactor inlet pressure <1435 mbar) and in the inventive tests (absolute reactor inlet pressure >1435 mbar) during process steps b) and c) is shown in FIG. 1 to FIG. 4.

In order to ascertain the phthalic anhydride yield and productivity during the catalytic tests, the product stream each case was analyzed with regard to its composition at regular intervals by means of a gas chromatograph (GC 7890B, Agilent) and a non-dispersive IR analyzer (NGA 2000, Rosemount).

Phthalic anhydride yield was calculated by equation Eq. 2.

$${\text{Y}}_{P A} = \left[139.52 - \left(800 * \frac{A + B}{B}\right) + \left(1.00 - F\right) - \left(1.25 * H\right) - \left(1.1 * G\right)\right]$$

A = CO₂ in product stream [% by vol.]
B = CO in product stream [% by vol.]
H = maleic anhydride content in product stream [% by wt.]
E = ortho-xylene loading in reactant stream [g/m³ (STP)]
F = purity of the ortho-xylene used [% by wt.]
G = ortho-xylene slip in product stream [% by wt.]
Y_{PA} = yield of phthalic anhydride (PA) based on the total weight of the ortho-xylene used [% by wt.]

As apparent from equation Eq. 2, the phthalic anhydride is directly dependent on the formation of the three most important by-products: CO, CO₂ and maleic anhydride.

Phthalic anhydride productivity was calculated by equation Eq. 3.

$${P}_{P A} = E * Q * \frac{{Y}_{P A}}{100 \% b y w t ,}$$

E = ortho-xylene loading in the reactant stream [g/m³ (STP)]
Q = air flow rate [m³ (STP)/h/tube]
Y_{PA} = phthalic anhydride (PA) yield based on the total weight of the ortho-xylene used [% by wt.]
P_{PA} = phthalic anhydride (PA) productivity [g/h/tube]

The progression of the phthalic anhydride yield and phthalic anhydride productivity over time for the inventive examples (absolute reactor inlet pressure >1435 mbar) and the noninventive comparative examples (absolute reactor inlet pressure < 1435 mbar) is shown in FIG. 5 to FIG. 8.

TABLE 1

Shaped catalyst bodies used

| Catal yst | Ring shape | Binder content | Proportion of active composition | BE T | TiO₂ | V₂O₅ | Promot ers⁵ |
|---|---|---|---|---|---|---|---|
| | (De x H x Di)¹ [mm] | [% by wt.]² | [% by wt.]³ | [m²/ g] | [% by wt.]⁴ | [% by wt.]⁴ | [% by wt.]⁵ |
| A0 | 8×6×5 | 2.4 | 9.5 | 18 | 87.8 | 7.5 | 4.7 |
| A1 | 6×5×4 | 2.4 | 5.6 | 26 | 82.7 | 11.0 | 6.3 |
| A2 | 8×6×5 | 2.4 | 8.7 | 18 | 87.9 | 7.5 | 4.6 |
| A3 | 8×6×5 | 2.3 | 8.4 | 25 | 89.4 | 9.4 | 1.2 |

¹ De = external diameter, H = height, Di = internal diameter
² based on the total weight of the shaped catalyst body
³ based on the total weight of the shaped catalyst body without binder
⁴ based on the active catalyst composition
⁵ including Sb₂O₃ and small proportions of Nb₂O₅, P and Cs

TABLE 2

Filling parameters for comparative test 1

| Catalyst layer | Catalyst | Fill heights | Bulk density | Ring shape |
|---|---|---|---|---|
| | | [cm] | [g/cm³] | (De x H x Di) |
| 1 | A0 | 43 | 0.80 | 8×6×5 |
| 2 | A1 | 151 | 0.87 | 6×5×4 |
| 3 | A2 | 70 | 0.86 | 8×6×5 |
| 4 | A3 | 71 | 0.82 | 8×6×5 |

TABLE 3

Filling parameters for comparative test 2

| Catalyst layer | Catalyst | Fill heights | Bulk density | Ring shape |
|---|---|---|---|---|
| | | [cm] | [g/cm³] | (De x H x Di) [mm] |
| 1 | A0 | 40 | 0.82 | 8×6×5 |
| 2 | A1 | 151 | 0.85 | 6×5×4 |
| 3 | A2 | 70 | 0.83 | 8×6×5 |
| 4 | A3 | 70 | 0.81 | 8×6×5 |

TABLE 4

Filling parameters for inventive test 1

| Catalyst layer | Catalyst | Fill heights | Bulk density | Ring shape |
|---|---|---|---|---|
| | | [cm] | [g/cm³] | (De x H x Di) [mm] |
| 1 | A0 | 40 | 0.82 | 8×6×5 |
| 2 | A1 | 150 | 0.87 | 6×5×4 |
| 3 | A2 | 71 | 0.81 | 8×6×5 |
| 4 | A3 | 71 | 0.81 | 8×6×5 |

TABLE 5

Filling parameters for inventive test 2

| Catalyst layer | Catalyst | Fill heights | Bulk density | Ring shape |
|---|---|---|---|---|
| | | [cm] | [g/cm³] | (De x H x Di) [mm] |
| 1 | A0 | 40 | 0.81 | 8×6×5 |
| 2 | A1 | 151 | 0.89 | 6×5×4 |
| 3 | A2 | 70 | 0.87 | 8×6×5 |
| 4 | A3 | 70 | 0.81 | 8×6×5 |

1. A process for starting up a reactor for preparation of phthalic anhydride by the catalytic oxidation of ortho-xylene and/or naphthalene, containing a bed of shaped catalyst bodies and within a temperature-controlled salt bath, comprising the steps of:
a) calcining the shaped catalyst bodies, in the presence of air and/or O2, at a salt bath temperature exceeding 390° C.,
b) adjusting the temperature of the salt bath to a temperature between 370° C. and 400° C.,
c) forming a hotspot in the front third of the catalyst bed in flow direction, by feeding in ortho-xylene and/or naphthalene,
d) cooling the salt bath temperature to a salt bath temperature below 360° C. at a rate of greater than 0.5° C./h and increasing the feed of ortho-xylene and/or naphthalene to a loading exceeding 70 g/m³ (STP), at an air flow rate of 2 m³ (STP)/h to 4.5 m³ (STP)/h,
wherein, during the feeding with ortho-xylene and/or naphthalene, the absolute reactor inlet pressure does not go below 1435 mbar.

2. The process as claimed in claim 1, wherein the maximum temperature of the shaped catalyst bodies during a) is always in the range between 390° C. and 460° C., preferably in the range between 400° C. and 440° C.

3. The process as claimed in claim 1, wherein step a) is effected for at least 6 h, more preferably at least 24 h.

4. The process as claimed in claim 1, wherein the salt bath temperature in step c) is kept stable between 370° C. and 400° C. for a period of between 1 h and 200 h.

5. The process as claimed in claim 1, wherein the ortho-xylene and/or naphthalene loading in step c) is between 10 g/m³ (STP) and 40 g/m³ (STP), at an air flow rate of 2 m³ (STP)/h to 4.5 m³ (STP)/h.

6. The process as claimed in claim 1, wherein the cooling in step d) is effected at a rate of > 0.70° C./h, preferably > 1° C./h.

7. The process as claimed in claim 1, wherein the cooling in d) is effected at a rate between 0.5 and 10° C./h, preferably 1° C./h to 10° C./h.

8. The process as claimed in claim 1, wherein the ortho-xylene and/or naphthalene loading on commencement of cooling in step d) is between 10 and 40 g/m³ (STP) and is increased in step d) to more than 70 g/m³ (STP), at an air flow rate of constantly between 2 m³ (STP)/h to 4.5 m³ (STP)/h.

9. The process as claimed in claim 1, wherein the ortho-xylene and/or naphthale0ne loading in each of steps c) or d) is only sufficiently high that the temperature of the shaped catalyst bodies does not exceed 455° C.

10. The process as claimed in claim 1, wherein the absolute reactor inlet pressure during steps b) and d) does not go below 1450 mbar, more preferably 1460 mbar.

11. The process as claimed in claim 1, wherein the reactor contains four or more catalyst layers consisting of different shaped catalyst bodies.

12. The process as claimed in claim 1, wherein one of the catalyst layers has a lower voidage than the other catalyst layers.

13. The process as claimed in claim 12, wherein the layer having the lower voidage has a voidage below 65%.
